(19) United States
(12) Patent Application Publication
(10) Pub. No.: US 2025/0282727 A1
(43) Pub. Date: Sep. 11, 2025

(54) Method for Preparing 2-chloro-5-methylpyridine by Continuous Flow

(71) Applicants: Jiangsu Ruixiang Chemical Co., Ltd., Yangzhou, Jiangsu (CN); Jiangsu Yangnong Chemical Group Co., Ltd., Yangzhou, Jiangsu (CN)

(72) Inventors: Yiming WANG, Yangzhou, Jiangsu (CN); Weiwei ZHANG, Yangzhou, Jiangsu (CN); Hui ZHAO, Yangzhou, Jiangsu (CN); Hui YAN, Yangzhou, Jiangsu (CN); Xia LIU, Yangzhou, Jiangsu (CN); Qiumin GAO, Yangzhou, Jiangsu (CN)

(21) Appl. No.: 18/860,649

(22) PCT Filed: Jan. 17, 2023
(86) PCT No.: PCT/CN2023/072699

§ 371 Date: Oct. 26, 2024

(30) Foreign Application Priority Data
Jul. 18, 2022 (CN) 202210842816.3

(51) Int. Cl. C07D 213/61

(52) U.S. Cl. CPC C07D 213/61

(57) ABSTRACT

The present disclosure relates to method for preparing 2-chloro-5-methylpyridine by continuous flow. The method includes the following steps: (1) pyridine oxide-organic nitrogen base homogeneous solution is mixed with chlorinating agent solution, to obtain salifying solution; and (2) the salifying solution is mixed with hydrogen chloride, to obtain chlorination reaction solution. A chlorinating agent in the chlorinating agent solution in Step (1) includes any one or a combination of at least two of phosgene, diphosgene, triphosgene, thionyl chloride, sulfuryl chloride, or cyanuric chloride. According to the preparation method, the hydrogen chloride is used for a chlorination reaction, and a pyridine oxide, an organic nitrogen base, and the chlorinating agent are mixed in the form of solution, so that compared with a traditional kettle-type intermittent synthesis method, the present disclosure improves the quality stability of a product in a synthesis process, and achieves high productivity and yield of the 2-chloro-5-methylpyridine.

### CROSS-REFERENCE TO RELATED APPLICATIONS

The present application is a National Stage of International Patent Application No. PCT/CN2023/072699 filed on Jan. 17, 2023, which claims priority to Chinese Application No. 202210842816.3, filed on Jul. 18, 2022, the disclosure of which is hereby incorporated again by reference in its entirety.

### TECHNICAL FIELD

The present disclosure belongs to the technical field of organic synthesis and relates to a synthesis method for a compound, in particular to method for preparing 2-chloro-5-methylpyridine by continuous flow.

### BACKGROUND

2-chloro-5-methylpyridine is an organic intermediate with higher application value, and widely used in fields such as pesticides, pharmaceuticals, and fine chemicals. It is a key intermediate for new-type and efficient pesticides imidacloprid and acetamiprid, and also a component with the most insecticidal activity in this class of pesticide molecules.

According to the classification of starting raw materials, synthesis methods for 2-chloro-5-methylpyridine include a 3-methylpyridine method, a pentene derivative method, and a propionaldehyde and nitrogen-containing organic compound method. The 3-methylpyridine synthesis method may generate a byproduct, 2-chloro-3-methylpyridine. However, with the development of separation technologies, high-purity 2-chloro-5-methylpyridine and 2-chloro-3-methylpyridine may be separated now, and 2-chloro-3-methylpyridine is also an important pesticide intermediate. Therefore, the synthesis of 2-chloro-5-methylpyridine from a 3-methylpyridine raw material by oxidation and chlorination is a reasonable process route.

U.S. Pat. No. 4,897,488 discloses a method for preparing 2-chloro-5-methylpyridine by starting from 3-methylpyridine oxide, using dichloromethane as a solvent, and reacting phosphorus oxychloride with triethylamine at −10° C. The highest yield may reach 81%, but this method may generate a large amount of difficult-to-treat phosphorus-containing wastewater in industrial production processes.

U.S. Pat. No. 5,010,201 discloses a method for preparing 2-chloro-5-methylpyridine by starting from 3-methylpyridine oxide, using diisopropylamine and dichloromethane as solvents, and using phosphoramide chloride as a chlorinating agent. However, the yield of this method is only 57%-68%, and preparation steps of the raw material phosphoramide chloride are cumbersome, so it is difficult to achieve the large-scale production.

The above traditional processes for synthesizing 2-chloro-5-methylpyridine from 3-methylpyridine oxide all use an intermittent kettle-type process for product synthesis, the process requires a large number of kettle-type reactors and low reaction temperatures (−5° C. to −10° C.). However, each step of the 3-methylpyridine oxide reaction is a strong exothermic reaction, the adiabatic temperature rise of the reaction exceeds 100° C., and deep-cold saltwater is required as a refrigerant medium. Therefore, the traditional intermittent kettle-type synthesis method is low in inherent safety and also has disadvantages such as higher refrigerant energy consumption, long reaction retention time, and low unit production capacity.

Therefore, in response to the shortcomings of existing technologies, it is necessary to provide a method for preparing 2-chloro-5-methylpyridine with high synthesis efficiency, low refrigerant consumption, and higher unit production capacity.

### SUMMARY

A purpose of the present disclosure is to provide method for preparing 2-chloro-5-methylpyridine by continuous flow. Compared to the traditional intermittent kettle-type synthesis method, the method is higher in synthesis efficiency, less in refrigerant consumption, higher in production capacity and yield of 2-chloro-5-methylpyridine, and higher in technical intrinsic safety.

In order to achieve the purpose of the present disclosure, the present disclosure adopts the following technical schemes.

The present disclosure provides a method for preparing 2-chloro-5-methylpyridine by a continuous flow, and the method includes the following steps.
(1) Pyridine oxide-organic nitrogen base homogeneous solution is mixed with chlorinating agent solution, to obtain salifying solution.
(2) The salifying solution is mixed with hydrogen chloride, to obtain chlorination reaction solution.

The main reaction equation of the preparation method provided by the present disclosure is as follows.

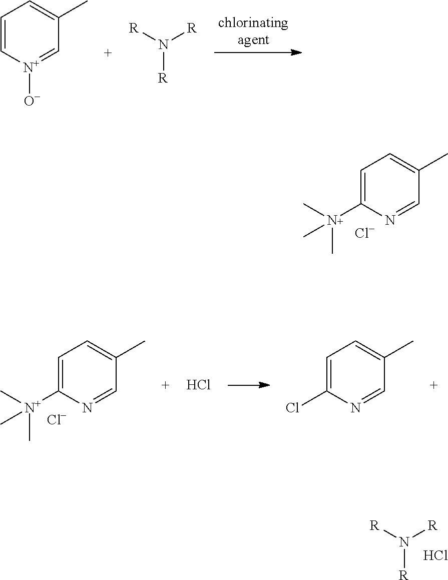

Herein, R represents alkane and/or aromatic hydrocarbon with a C atom number ≥1, for example it may be any one or a combination of at least two of methane, ethane, propane, or butane. Typical but non-restrictive combinations include a combination of methane and ethane, a combination of ethane and propane, a combination of propane and butane, a combination of methane, ethane and propane, or a combination of methane, ethane, propane and butane.

The method provided by the present disclosure uses hydrogen chloride for a chlorination reaction, and mixes pyridine oxide, organic nitrogen base, and chlorinating agent in the form of solution, as to improve the quality stability of a product during the synthesis process, and the production capacity and yield of 2-chloro-5-methylpyridine are relatively high.

Preferably, a chlorinating agent in the chlorinating agent solution in Step (1) includes any one or a combination of at least two of phosgene, diphosgene, triphosgene, thionyl chloride, sulfuryl chloride, or cyanuric chloride. Typical but non-restrictive combinations include a combination of phosgene and diphosgene, a combination of triphosgene and thionyl chloride, a combination of sulfuryl chloride and cyanuric chloride, a combination of phosgene, diphosgene and triphosgene, a combination of thionyl chloride, sulfuryl chloride and cyanuric chloride, a combination of phosgene, diphosgene, sulfuryl chloride and cyanuric chloride, or a combination of phosgene, diphosgene, triphosgene, thionyl chloride, sulfuryl chloride and cyanuric chloride, preferably phosgene and/or triphosgene.

Preferably, a pyridine oxide in the pyridine oxide-organic nitrogen base homogeneous solution in Step (1) includes 3-methylpyridine oxide.

Preferably, an organic nitrogen base in the pyridine oxide-organic nitrogen base homogeneous solution in Step (1) comprises any one or a combination of at least one of trimethylamine, triethylamine, tripropylamine, tributylamine, N,N-dimethylbenzylamine, or diisopropylamine. Typical but non-restrictive combinations include a combination of trimethylamine and triethylamine, a combination of tripropylamine and tributylamine, a combination of N,N-dimethylbenzylamine and diisopropylamine, a combination of trimethylamine, triethylamine and tripropylamine, a combination of tripropylamine, tributylamine and N,N-dimethylbenzylamine, a combination of trimethylamine, triethylamine, tripropylamine and diisopropylamine, or a combination of f trimethylamine, triethylamine, tripropylamine, tributylamine, N,N-dimethylbenzylamine and diisopropylamine, preferably trimethylamine.

Preferably, a solvent in the pyridine oxide-organic nitrogen base homogeneous solution in Step (1) includes any one or a combination of at least two of dichloromethane, chloroform, dichloroethane, chlorobenzene or dichlorobenzene. Typical but non-restrictive combinations include a combination of dichloromethane and chloroform, a combination of chloroform and dichloroethane, a combination of chlorobenzene and dichlorobenzene, a combination of dichloromethane, chloroform and dichloroethane, a combination of chloroform, chlorobenzene and dichlorobenzene, a combination of dichloromethane, chloroform, dichloroethane and chlorobenzene, or a combination of dichloromethane, chloroform, dichloroethane, chlorobenzene and dichlorobenzene, preferably dichloromethane.

Preferably, a solvent in the chlorinating agent solution in Step (1) includes any one or a combination of at least two of dichloromethane, chloroform, dichloroethane, chlorobenzene, or dichlorobenzene. Typical but non-restrictive combinations include a combination of dichloromethane and chloroform, a combination of chloroform and dichloroethane, a combination of chlorobenzene and dichlorobenzene, a combination of dichloromethane, chloroform and dichloroethane, a combination of chloroform, chlorobenzene and dichlorobenzene, a combination of dichloromethane, chloroform, dichloroethane and chlorobenzene, or a combination of dichloromethane, chloroform, dichloroethane, chlorobenzene and dichlorobenzene, preferably dichloromethane.

Preferably, the solvent in the pyridine oxide-organic nitrogen base homogeneous solution in Step (1) is the same as the solvent in the chlorinating agent solution.

Preferably, in the pyridine oxide-organic nitrogen base homogeneous solution in Step (1), the concentration of the pyridine oxide is 1 wt %-20 wt %, for example it may be 1 wt %, 3 wt %, 5 wt %, 6 wt %, 8 wt %, 10 wt %, 12 wt %, 15 wt %, 16 wt %, 18 wt % or 20 wt %, but not limited to the values listed. Other values unlisted within the numerical range are also applicable, preferably 5 wt %-15 wt %.

Preferably, in the pyridine oxide-organic nitrogen base homogeneous solution in Step (1), the molar ratio of the organic nitrogen base to the pyridine oxide is (1-4):1, for example it may be 1:1, 1.5:1, 2:1, 2.5:1, 3:1, 3.5:1 or 4:1, but not limited to the values listed. Other values unlisted within the numerical range are also applicable, preferably (2-3):1.

Preferably, the concentration of the chlorinating agent solution in Step (1) is 10 wt %-50 wt %, for example it may be 10 wt %, 15 wt %, 20 wt %, 25 wt %, 30 wt %, 35 wt %, 40 wt %, 45 wt %, or 50 wt %, but not limited to the values listed. Other values unlisted within the numerical range are also applicable, preferably 20 wt %-30 wt %.

Preferably, in the salifying solution in Step (1), the molar ratio of the chlorinating agent to the pyridine oxide is (0.1-3): 1, for example it may be 0.1:1, 0.3:1, 0.5:1, 0.6:1, 0.8:1, 0.9:1, 1:1, 1.2:1, 1.5:1, 1.8:1, 2:1, 2.1:1, 2.4:1, 2.5:1, 2.7:1, 2.8:1, or 3:1, but not limited to the values listed. Other values unlisted within the numerical range are also applicable, preferably (0.9-1.5):1.

Preferably, the molar ratio of the hydrogen chloride to the pyridine oxide in Step (2) is 1: (1-8), for example it may be 1:1, 1:2, 1:3, 1:4, 1:5, 1:6, 1:7, or 1:8, but not limited to the values listed. Other values unlisted within the numerical range are also applicable, preferably 1: (3-5).

Preferably, the temperature of the mixing in Step (1) is 10° C.-100° C., for example it may be 10° C., 20° C., 30° C., 40° C., 50° C., 60° C., 70° C., 80° C., 90° C., or 100° C., but not limited to the values listed. Other values unlisted within the numerical range are also applicable, preferably 30° C.-50° C.

Preferably, the time of the mixing in Step (1) is 5 s-30 s, for example it may be 5 s, 7 s, 10 s, 15 s, 20 s, 25 s, or 30 s, but not limited to the values listed. Other values unlisted within the numerical range are also applicable, preferably 7 s-20 s.

Preferably, the temperature of the mixing in Step (2) is 10° C.-200° C., for example it may be 10° C., 30° C., 50° C., 60° C., 80° C., 100° C., 110° C., 120° C., 130° C., 140° C., 150° C., 160° C., 180° C. or 200° C., but not limited to the values listed. Other values unlisted within the numerical range are also applicable, preferably 110° C.-130° C.

Preferably, the system pressure during the mixing in Step (2) is 0.2 MPa-3 MPa, for example it may be 0.2 MPa, 0.4 MPa, 0.5 MPa, 0.6 MPa, 0.8 MPa, 1 MPa, 1.2 MPa, 1.5 MPa, 1.6 MPa, 1.8 MPa, 2 MPa, 2.5 MPa, or 3 MPa, but not limited to the values listed. Other values unlisted within the numerical range are also applicable, preferably 0.3 MPa-1 MPa.

Preferably, the hydrogen chloride in Step (2) of the present disclosure is a hydrogen chloride gas. By controlling a pressure reducing valve of a hydrogen chloride supply apparatus and an outlet back pressure valve of a device for mixing, it is possible to regulate the system pressure during the mixing, thereby the pressure of the mixing in Step (2) is controlled to 0.2 MPa-3 MPa.

Preferably, the time of the mixing in Step (2) is 30 s-300 s, for example it may be 30 s, 40 s, 50 s, 60 s, 80 s, 100 s, 120 s, 150 s, 160 s, 180 s, 200 s, 210 s, 240 s, 250 s, 270 s, 280 s, or 300 s, but not limited to the values listed. Other values unlisted within the numerical range are also applicable, preferably 150 s-200 s.

Preferably, the mixing in Step (1) is performed in a first microchannel reactor.

As a preferred technical scheme provided by the present disclosure, when the mixing in Step (1) is performed in the first microchannel reactor, the time of the mixing in Step (1) is the retention time of the material in the first microchannel reactor.

Preferably, the effective volume of the first microchannel reactor is 1 mL-200 mL, for example it may be 1 mL, 5 mL, 10 mL, 20 mL, 30 mL, 40 mL, 50 mL, 60 mL, 70 mL, 80 mL, 100 mL, 120 mL, 150 mL, 160 mL, 180 mL or 200 mL, but not limited to the values listed. Other values unlisted within the numerical range are also applicable, preferably 5 mL-50 mL.

Preferably, the mixing in Step (2) is performed in a second microchannel reactor.

As a preferred technical scheme provided by the present disclosure, when the mixing in Step (2) is performed in the second microchannel reactor, the time of the mixing in Step (2) is the retention time of the material in the second microchannel reactor.

Preferably, the effective volume of the second microchannel reactor is 1 mL-200 mL, for example it may be 1 mL, 5 mL, 10 mL, 20 mL, 30 mL, 40 mL, 50 mL, 60 mL, 70 mL, 80 mL, 100 mL, 120 mL, 150 mL, 160 mL, 180 mL or 200 mL, but not limited to the values listed. Other values unlisted within the numerical range are also applicable, preferably 5 mL-50 mL.

The present disclosure does not limit the specific models of the first microchannel reactor and the second microchannel reactor, as long as the effective volume is 1 mL-200 mL and the retention time of the material is conformed to the mixing requirements.

The traditional intermittent kettle-type process has disadvantages such as high heat release, low intrinsic safety, high refrigerant energy consumption, high catalyst and chlorinating agent dosage, long retention time, and low reaction yield. In the present disclosure, the mixing of the pyridine oxide-organic nitrogen base homogeneous solution and the chlorinating agent solution, as well as the mixing of the hydrogen chloride and the salifying solution, are performed in the microchannel reactor, which may utilize the strong heat transfer ability of the microchannel reactor to increase the mixing temperature in Step (1) by 40° C.-50° C. on the basis of the traditional intermittent kettle-type process. The reaction temperature is more suitable and the heat transfer energy consumption may be significantly reduced; at the same time, the mixing effect of the material in the microchannel reactor is good, and there is no backmixing phenomenon, which may effectively suppress the occurrence of side reactions, reduce the generation of isomer 2-chloro-3-methylpyridine, and improve the yield and selectivity of the main product; reacting in the microchannel reactor also has the characteristics of simple process flow, and high unit production capacity, and is beneficial for industrial transformation and promotion; and furthermore, due to the small liquid holdup of the reactor, it may significantly reduce reaction risks and improve the intrinsic safety of the production process.

In the present disclosure, when the first microchannel reactor is used for mixing in Step (1), the pyridine oxide-organic nitrogen base homogeneous solution and the chlorinating agent solution are respectively fed into the first microchannel reactor by a conventional conveying apparatus in this field for a mixing reaction. After a certain retention time, the salifying solution is obtained.

In the present disclosure, when the second microchannel reactor is used for mixing in Step (2), the salifying solution and the hydrogen chloride gas are fed into the second microchannel reactor by the conventional conveying apparatus in this field. The flow rate of the hydrogen chloride gas is controlled by a pressure reducing valve and a mass flow meter, and the system pressure in the second microchannel reactor is controlled by a back pressure valve. After a certain retention time, the chlorination reaction solution is obtained.

As a preferred technical scheme of the method in the present disclosure, the method includes the following steps.
(1) In the first microchannel reactor with the effective volume of 1 mL-200 mL, pyridine oxide-organic nitrogen base homogeneous solution is mixed with chlorinating agent solution at 10° C.-100° C., to obtain salifying solution, herein the retention time is 5 s-30 s.
(2) In the second microchannel reactor with the effective volume of 1 mL-200 mL, the salifying solution is mixed with hydrogen chloride at 10° C.-200° C., to obtain chlorination reaction solution, herein the system pressure is 0.2 MPa-3 MPa, and the retention time is 30 s-300 s.

A chlorinating agent in the chlorinating agent solution in Step (1) includes any one or a combination of at least two of phosgene, diphosgene, triphosgene, thionyl chloride, sulfuryl chloride, or cyanuric chloride; the concentration of the chlorinating agent solution is 10 wt %-50wt %.

A pyridine oxide in the pyridine oxide-organic nitrogen base homogeneous solution in Step (1) includes 3-methylpyridine oxide, and the concentration is 1 wt %-20wt %; an organic nitrogen base includes any one or a combination of at least one of trimethylamine, triethylamine, tripropylamine, tributylamine, N,N-dimethylbenzylamine, or diisopropylamine; the molar ratio of the organic nitrogen base to the pyridine oxide is (1-4):1.

A solvent in the pyridine oxide-organic nitrogen base homogeneous solution in Step (1) includes any one or a combination of at least two of dichloromethane, chloroform, dichloroethane, chlorobenzene or dichlorobenzene.

In the salifying solution in Step (1), the molar ratio of the chlorinating agent to the pyridine oxide is (0.1-3):1.

The molar ratio of the hydrogen chloride to the pyridine oxide in Step (2) is 1: (1-8).

Compared to existing technologies, the present disclosure has the following beneficial effects.
(1) The method provided by the present disclosure uses the hydrogen chloride for the chlorination reaction, and mixes the pyridine oxide, the organic nitrogen base, and the chlorinating agent in the form of solution, which improves the stability of product quality during the synthesis process, and has the higher product production capacity and yield.
(2) As a further preferred technical effect, in the present disclosure, the mixing of the pyridine oxide-organic nitrogen base homogeneous solution and the chlorinating agent solution, as well as the mixing of the hydrogen chloride and the salifying solution, are performed in the microchannel reactor, which may utilize the strong heat transfer ability of the microchannel reactor to increase the mixing temperature by 40° C.-50° C. on the basis of the traditional intermittent kettle-type process, the reaction temperature is more suitable and the heat transfer energy consumption may be significantly reduced; at the same time, the mixing effect of the material in the microchannel reactor is good, and there is no backmixing phenomenon, which may effectively suppress the occurrence of side reactions, reduce the generation of isomer 2-chloro-3-methylpyridine, and improve the yield and selectivity of the main product; reacting in the microchannel reactor also has the characteristics of simple process flow, and high unit production capacity, and is beneficial for industrial transformation and promotion; and furthermore, due to the small liquid holdup of the reactor, it may significantly reduce reaction risks and improve the intrinsic safety of the production process.

### DETAILED DESCRIPTION OF THE EMBODIMENTS

Technical schemes of the present disclosure are further described below by specific embodiments. Those skilled in the art should understand that embodiments are only intended to assist in understanding the present disclosure and should not be considered as specific limitations on the present disclosure.

### Embodiment 1

This embodiment provided a method for preparing 2-chloro-5-methylpyridine by a continuous flow, and the method included the following steps.
(1) In a first microchannel reactor with an effective volume of 30 mL, pyridine oxide-organic nitrogen base homogeneous solution was mixed with chlorinating agent solution at 40° C., to obtain salifying solution, and the retention time was 15 s; when the first microchannel reactor was used for mixing, the pyridine oxide-organic nitrogen base homogeneous solution and the chlorinating agent solution were respectively fed into the first microchannel reactor by a metering pump for a mixing reaction, and after a certain retention time, the salifying solution was obtained.
(2) In a second microchannel reactor with an effective volume of 30 mL, the salifying solution was mixed with hydrogen chloride at 120° C. and a system pressure of 0.6 MPa, to obtain chlorination reaction solution, and the retention time was 180 s; when the second microchannel reactor was used for mixing, the salifying solution and the hydrogen chloride gas were fed into the second microchannel reactor by the metering pump, the flow rate of the hydrogen chloride gas was controlled by a pressure reducing valve and a mass flow meter, the system pressure in the second microchannel reactor was controlled by a back pressure valve, and after a certain retention time, the chlorination reaction solution was obtained.

A chlorinating agent in the chlorinating agent solution in Step (1) was phosgene; the concentration of the chlorinating agent solution was 25 wt %; a solvent in the chlorinating agent solution was dichloromethane.

A pyridine oxide in the pyridine oxide-organic nitrogen base homogeneous solution in Step (1) was 3-methylpyridine oxide, and the concentration was 9.1 wt %; the organic nitrogen base was trimethylamine; the molar ratio of the organic nitrogen base to the pyridine oxide was 2.5:1; a solvent in the pyridine oxide-organic nitrogen base homogeneous solution in Step (1) was dichloromethane.

In the salifying solution in Step (1), the molar ratio of the chlorinating agent to the pyridine oxide was 1.2:1.

The molar ratio of the hydrogen chloride to the pyridine oxide in Step (2) was 1:4.

### Embodiment 2

This embodiment provided a method for preparing 2-chloro-5-methylpyridine by a continuous flow, and the method included the following steps.
(1) In a first microchannel reactor with an effective volume of 50 mL, pyridine oxide-organic nitrogen base homogeneous solution was mixed with chlorinating agent solution at 30° C., to obtain salifying solution, and the retention time was 20 s; when the first microchannel reactor was used for mixing, the pyridine oxide-organic nitrogen base homogeneous solution and the chlorinating agent solution were respectively fed into the first microchannel reactor by a metering pump for a mixing reaction, and after a certain retention time, the salifying solution was obtained.
(2) In a second microchannel reactor with an effective volume of 50 mL, the salifying solution was mixed with hydrogen chloride at 110° C. and a system pressure of 1 MPa, to obtain chlorination reaction solution, and the retention time was 200 s; when the second microchannel reactor was used for mixing, the salifying solution and the hydrogen chloride gas were fed into the second microchannel reactor by the metering pump, the flow rate of the hydrogen chloride gas was controlled by a pressure reducing valve and a mass flow meter, the system pressure in the second microchannel reactor was controlled by a back pressure valve, and after a certain retention time, the chlorination reaction solution was obtained.

A chlorinating agent in the chlorinating agent solution in Step (1) was phosgene; the concentration of the chlorinating agent solution was 20 wt %; a solvent in the chlorinating agent solution was dichloromethane.

A pyridine oxide in the pyridine oxide-organic nitrogen base homogeneous solution in Step (1) was 3-methylpyridine oxide, and the concentration was 5 wt %; the organic nitrogen base was trimethylamine; the molar ratio of the organic nitrogen base to the pyridine oxide was 3:1; a solvent in the pyridine oxide-organic nitrogen base homogeneous solution in Step (1) was dichloromethane.

In the salifying solution in Step (1), the molar ratio of the chlorinating agent to the pyridine oxide was 0.9:1.

The molar ratio of the hydrogen chloride to the pyridine oxide in Step (2) was 1:3.

### Embodiment 3

This embodiment provided a method for preparing 2-chloro-5-methylpyridine by a continuous flow, and the method included the following steps.
(1) In a first microchannel reactor with an effective volume of 5 mL, pyridine oxide-organic nitrogen base homogeneous solution was mixed with chlorinating agent solution at 50° C., to obtain salifying solution, and the retention time was 7 s; when the first microchannel reactor was used for mixing, the pyridine oxide-organic nitrogen base homogeneous solution and the chlorinating agent solution were respectively fed into the first microchannel reactor by a metering pump for a mixing reaction, and after a certain retention time, the salifying solution was obtained.
(2) In a second microchannel reactor with an effective volume of 5 mL, the salifying solution was mixed with hydrogen chloride at 130° C. and a system pressure of 0.3 MPa, to obtain chlorination reaction solution, and the retention time was 150 s; when the second microchannel reactor was used for mixing, the salifying solution and the hydrogen chloride gas were fed into the second microchannel reactor by the metering pump, the flow rate of the hydrogen chloride gas was controlled by a pressure reducing valve and a mass flow meter, the system pressure in the second microchannel reactor was controlled by a back pressure valve, and after a certain retention time, the chlorination reaction solution was obtained.

A chlorinating agent in the chlorinating agent solution in Step (1) was phosgene; the concentration of the chlorinating agent solution was 30 wt %; a solvent in the chlorinating agent solution was dichloromethane.

A pyridine oxide in the pyridine oxide-organic nitrogen base homogeneous solution in Step (1) was 3-methylpyridine oxide, and the concentration was 15 wt %; the organic nitrogen base was trimethylamine; the molar ratio of the organic nitrogen base to the pyridine oxide was 2:1; a solvent in the pyridine oxide-organic nitrogen base homogeneous solution in Step (1) was dichloromethane.

In the salifying solution in Step (1), the molar ratio of the chlorinating agent to the pyridine oxide was 1.5:1.

The molar ratio of the hydrogen chloride to the pyridine oxide in Step (2) was 1:5.

### Embodiment 4

This embodiment provided a method for preparing 2-chloro-5-methylpyridine by a continuous flow, and the method included the following steps.
(1) In a first microchannel reactor with an effective volume of 200 mL, pyridine oxide-organic nitrogen base homogeneous solution was mixed with chlorinating agent solution at 10° C., to obtain salifying solution, and the retention time was 30 s; when the first microchannel reactor was used for mixing, the pyridine oxide-organic nitrogen base homogeneous solution and the chlorinating agent solution were respectively fed into the first microchannel reactor by a metering pump for a mixing reaction, and after a certain retention time, the salifying solution was obtained.
(2) In a second microchannel reactor with an effective volume of 200 mL, the salifying solution was mixed with hydrogen chloride at 10° C. and a system pressure of 3 MPa, to obtain chlorination reaction solution, and the retention time was 300 s; when the second microchannel reactor was used for mixing, the salifying solution and the hydrogen chloride gas were fed into the second microchannel reactor by the metering pump, the flow rate of the hydrogen chloride gas was controlled by a pressure reducing valve and a mass flow meter, the system pressure in the second microchannel reactor was controlled by a back pressure valve, and after a certain retention time, the chlorination reaction solution was obtained.

A chlorinating agent in the chlorinating agent solution in Step (1) was phosgene; the concentration of the chlorinating agent solution was 10 wt %; a solvent in the chlorinating agent solution was dichloromethane;

A pyridine oxide in the pyridine oxide-organic nitrogen base homogeneous solution in Step (1) was 3-methylpyridine oxide, and the concentration was 1 wt %; the organic nitrogen base was trimethylamine; the molar ratio of the organic nitrogen base to the pyridine oxide was 4:1; a solvent in the pyridine oxide-organic nitrogen base homogeneous solution in Step (1) was dichloromethane.

In the salifying solution in Step (1), the molar ratio of the chlorinating agent to the pyridine oxide was 0.1:1.

The molar ratio of the hydrogen chloride to the pyridine oxide in Step (2) was 1:1.

### Embodiment 5

This embodiment provided a method for preparing 2-chloro-5-methylpyridine by a continuous flow, and the method included the following steps.
(1) In a first microchannel reactor with an effective volume of 1 mL, pyridine oxide-organic nitrogen base homogeneous solution was mixed with chlorinating agent solution at 100° C., to obtain salifying solution, and the retention time was 5 s; when the first microchannel reactor was used for mixing, the pyridine oxide-organic nitrogen base homogeneous solution and the chlorinating agent solution were respectively fed into the first microchannel reactor by a metering pump for a mixing reaction, and after a certain retention time, the salifying solution was obtained.
(2) In a second microchannel reactor with an effective volume of 1 mL, the salifying solution was mixed with hydrogen chloride at 200° C. and a system pressure of 0.2 MPa, to obtain chlorination reaction solution, and the retention time was 30 s; when the second microchannel reactor was used for mixing, the salifying solution and the hydrogen chloride gas were fed into the second microchannel reactor by the metering pump, the flow rate of the hydrogen chloride gas was controlled by a pressure reducing valve and a mass flow meter, the system pressure in the second microchannel reactor was controlled by a back pressure valve, and after a certain retention time, the chlorination reaction solution was obtained.

A chlorinating agent in the chlorinating agent solution in Step (1) was phosgene; the concentration of the chlorinating agent solution was 50 wt %; a solvent in the chlorinating agent solution was dichloromethane.

A pyridine oxide in the pyridine oxide-organic nitrogen base homogeneous solution in Step (1) was 3-methylpyridine oxide, and the concentration was 20 wt %; the organic nitrogen base was trimethylamine; the molar ratio of the organic nitrogen base to the pyridine oxide was 1:1; a solvent in the pyridine oxide-organic nitrogen base homogeneous solution in Step (1) was dichloromethane.

In the salifying solution in Step (1), the molar ratio of the chlorinating agent to the pyridine oxide was 3:1.

The molar ratio of the hydrogen chloride to the pyridine oxide in Step (2) was 1:8.

### Embodiment 6

This embodiment provided a method for preparing 2-chloro-5-methylpyridine by a continuous flow, which was the same as Embodiment 1 except that the solvent used in the pyridine oxide-organic nitrogen base homogeneous solution and the solvent used in the chlorinating agent solution were dichloroethane.

### Embodiment 7

This embodiment provided a method for preparing 2-chloro-5-methylpyridine by a continuous flow, which was the same as Embodiment 1 except that the solvent used in the pyridine oxide-organic nitrogen base homogeneous solution and the solvent used in the chlorinating agent solution were chlorobenzene.

### Embodiment 8

This embodiment provided a method for preparing 2-chloro-5-methylpyridine by a continuous flow, which was the same as Embodiment 1 except that the solvent used in the pyridine oxide-organic nitrogen base homogeneous solution was dichloromethane and the solvent used in the chlorinating agent solution was dichloroethane.

### Embodiment 9

This embodiment provided a method for preparing 2-chloro-5-methylpyridine by a continuous flow, which was the same as Embodiment 1 except that the chlorinating agent was triphosgene.

### Embodiment 10

This embodiment provided a method for preparing 2-chloro-5-methylpyridine by a continuous flow, which was the same as Embodiment 1 except that the chlorinating agent was sulfuryl chloride.

### Embodiment 11

This embodiment provided a method for preparing 2-chloro-5-methylpyridine by a continuous flow, which was the same as Embodiment 1 except that the chlorinating agent was cyanuric chloride.

### Embodiment 12

This embodiment provided a method for preparing 2-chloro-5-methylpyridine by a continuous flow, which was the same as Embodiment 1 except that the organic nitrogen base was triethylamine.

### Embodiment 13

This embodiment provided a method for preparing 2-chloro-5-methylpyridine by a continuous flow, which was the same as Embodiment 1 except that the organic nitrogen base was tributylamine.

### Embodiment 14

This embodiment provided a method for preparing 2-chloro-5-methylpyridine by a continuous flow, which was the same as Embodiment 1 except that the organic nitrogen base was N,N-dimethylbenzylamine.

### Contrast Embodiment 1

This contrast embodiment provided a method for preparing 2-chloro-5-methylpyridine by a continuous flow, which was the same as Embodiment 1 except that the hydrogen chloride was replaced with an equimolar amount of phosphorus trichloride.

The chlorination reaction solution obtained from the above embodiments and contrast embodiment was subjected to conventional post-treatment processes such as neutralization, liquid separation, extraction, and desolvation in this field to obtain 2-chloro-5-methylpyridine. The yield of 2-chloro-5-methylpyridine was calculated by using the pyridine oxide as a raw material, and results were shown in Table 1.

TABLE 1

| | Yield of 2-chloro-5-methylpyridine |
|---|---|
| | (%) |
| Embodiment 1 | 92.9 |
| Embodiment 2 | 91.7 |
| Embodiment 3 | 92.4 |
| Embodiment 4 | 91.4 |
| Embodiment 5 | 89.5 |
| Embodiment 6 | 90.8 |
| Embodiment 7 | 91.2 |
| Embodiment 8 | 91.3 |
| Embodiment 9 | 91.1 |
| Embodiment 10 | 90.7 |
| Embodiment 11 | 91.4 |
| Embodiment 12 | 90.6 |
| Embodiment 13 | 91.2 |
| Embodiment 14 | 90.4 |
| Contrast embodiment 1 | 80.7 |

In conclusion, the method provided by the present disclosure used the hydrogen chloride for the chlorination reaction, and mixed the pyridine oxide, the organic nitrogen base, and the chlorinating agent in the form of solution, which improved the stability of product quality during the synthesis process, and had the higher product production capacity and yield; in the present disclosure, the mixing of the pyridine oxide-organic nitrogen base homogeneous solution and the chlorinating agent solution, as well as the mixing of the hydrogen chloride and the salifying solution, were performed in the microchannel reactor, which might utilize the strong heat transfer ability of the microchannel reactor to increase the mixing temperature by 40° C.-50° C. on the basis of the traditional intermittent kettle-type process, the reaction temperature was more suitable and the heat transfer energy consumption might be significantly reduced; at the same time, the mixing effect of the material in the microchannel reactor was good, and there was no backmixing phenomenon, which might effectively suppress the occurrence of side reactions, reduced the generation of isomer 2-chloro-3-methylpyridine, and improved the yield and selectivity of the main product; reacting in the microchannel reactor also had the characteristics of simple process flow, and high unit production capacity, and was beneficial for industrial transformation and promotion; and furthermore, due to the small liquid holdup of the reactor, it might significantly reduce reaction risks and improved the intrinsic safety of the production process.

The above are only the specific embodiments of the present disclosure, but the scope of protection of the present disclosure is not limited thereto. Those skilled in the art should understand that any changes or replacements that may be easily thought of by those skilled in the art within the technical scope disclosed by the present disclosure shall fall within the scope of protection and disclosure of the present disclosure.

1. A method for preparing 2-chloro-5-methylpyridine by a continuous flow, wherein the method comprises the following steps:
(1) mixing pyridine oxide-organic nitrogen base homogeneous solution with chlorinating agent solution, to obtain salifying solution; and
(2) mixing the salifying solution with hydrogen chloride, to obtain chlorination reaction solution.

2. The method according to claim 1, wherein a chlorinating agent in the chlorinating agent solution in Step (1) comprises any one or a combination of at least two of phosgene, diphosgene, triphosgene, thionyl chloride, sulfuryl chloride, or cyanuric chloride.

3. The method according to claim 1, wherein a solvent in the pyridine oxide-organic nitrogen base homogeneous solution in Step (1) comprises any one or a combination of at least two of dichloromethane, chloroform, dichloroethane, chlorobenzene or dichlorobenzene, preferably the dichloromethane.

4. The method according to claim 1, wherein in the pyridine oxide-organic nitrogen base homogeneous solution in Step (1), the concentration of the pyridine oxide is 1 wt %-20 wt %, preferably 5 wt %-15 wt %;
preferably, in the pyridine oxide-organic nitrogen base homogeneous solution in Step (1), the molar ratio of the organic nitrogen base to the pyridine oxide is (1-4):1, preferably (2-3):1.

5. The method according to claim 1, wherein the molar ratio of the hydrogen chloride to the pyridine oxide in Step (2) is 1: (1-8), preferably 1:(3-5).

6. The method according to claim 1, wherein the temperature of the mixing in Step (1) is 10° C.-100° C., preferably 30° C.-50° C.

7. The method according to claim 1, wherein the temperature of the mixing in Step (2) is 10° C.-200° C., preferably 110° C.-130° C.

8. The method according to claim 1, wherein the mixing in Step (1) is performed in a first microchannel reactor;
preferably, the effective volume of the first microchannel reactor is 1 mL-200 mL, preferably 5 mL-50 mL.

9. The method according to claim 1, wherein the mixing in Step (2) is performed in a second microchannel reactor;
preferably, the effective volume of the second microchannel reactor is 1 mL-200 mL, preferably 5 mL-50 mL.

10. The method according to claim 1, wherein the method comprises the following steps:
(1) in the first microchannel reactor with the effective volume of 1 mL-200 mL, mixing pyridine oxide-organic nitrogen base homogeneous solution with chlorinating agent solution at 10° C.-100° C., to obtain salifying solution, wherein the retention time is 5 s-30 s;
(2) in the second microchannel reactor with the effective volume of 1 mL-200 mL, mixing the salifying solution with hydrogen chloride at 10° C.-200° C., to obtain chlorination reaction solution, wherein the system pressure is 0.2 MPa-3 MPa, and the retention time is 30 s-300 s;
a chlorinating agent in the chlorinating agent solution in Step (1) comprises any one or a combination of at least two of phosgene, diphosgene, triphosgene, thionyl chloride, sulfuryl chloride, or cyanuric chloride; the concentration of the chlorinating agent solution is 10 wt %-50 wt %;
a pyridine oxide in the pyridine oxide-organic nitrogen base homogeneous solution in Step (1) comprises 3-methylpyridine oxide, and the concentration is 1 wt %-20 wt %; an organic nitrogen base comprises any one or a combination of at least one of trimethylamine, triethylamine, tripropylamine, tributylamine, N,N-dimethylbenzylamine, or diisopropylamine; the molar ratio of the organic nitrogen base to the pyridine oxide is (1-4):1;
a solvent in the pyridine oxide-organic nitrogen base homogeneous solution in Step (1) comprises any one or a combination of at least two of dichloromethane, chloroform, dichloroethane, chlorobenzene or dichlorobenzene;
in the salifying solution in Step (1), the molar ratio of the chlorinating agent to the pyridine oxide is (0.1-3):1;
the molar ratio of the hydrogen chloride to the pyridine oxide in Step (2) is 1: (1-8).

11. The method according to claim 1, wherein a pyridine oxide in the pyridine oxide-organic nitrogen base homogeneous solution in Step (1) comprises 3-methylpyridine oxide.

12. The method according to claim 1, wherein an organic nitrogen base in the pyridine oxide-organic nitrogen base homogeneous solution in Step (1) comprises any one or a combination of at least one of trimethylamine, triethylamine, tripropylamine, tributylamine, N,N-dimethylbenzylamine, or diisopropylamine, preferably the trimethylamine.

13. The method according to claim 1, wherein the chlorinating agent in the chlorinating agent solution in Step (1) comprises phosgene and/or triphosgene.

14. The method according to claim 1, wherein a solvent in the chlorinating agent solution in Step (1) comprises any one or a combination of at least two of dichloromethane, chloroform, dichloroethane, chlorobenzene, or dichlorobenzene, preferably the dichloromethane.

15. The method according to claim 1, wherein the solvent in the pyridine oxide-organic nitrogen base homogeneous solution in Step (1) is the same as the solvent in the chlorinating agent solution.

16. The method according to claim 1, wherein the concentration of the chlorinating agent solution in Step (1) is 10 wt %-50 wt %, preferably 20 wt %-30 wt %.

17. The method according to claim 1, wherein in the salifying solution in Step (1), the molar ratio of the chlorinating agent to the pyridine oxide is (0.1-3):1, preferably (0.9-1.5):1.

18. The method according to claim 1, wherein the time of the mixing in Step (1) is 5 s-30 s, preferably 7 s-20 s.

19. The method according to claim 1, wherein the system pressure during the mixing in Step (2) is 0.2 MPa-3 MPa, preferably 0.3 MPa-1 MPa.

20. The method according to claim 1, wherein the time of the mixing in Step (2) is 30 s-300 s, preferably 150 s-200 s.
